SEQUENCE LISTING (24 sequences)

SEQ ID NO: 1 - DNA, 323 bp, synthetic construct
  source 1..323: mol_type = other DNA
acccctctccagccactaagccagttgctccctcggctgacggctgcacgcgaggcctccgaacgtcttacgccttgtggcgcgcccgtccttgtcccggggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccg

SEQ ID NO: 2 - protein, 21 aa, synthetic construct
MALPVTALLLPLALLLHAARP

SEQ ID NO: 3 - DNA, 63 bp, synthetic construct
  source 1..63: mol_type = other DNA
atggcgcttcctgtcactgcacttttgctgccactggctttgcttcttcatgccgcaagaccc

SEQ ID NO: 4 - protein, 28 aa, synthetic construct
FWVLVVVGGVLACYSLLVTVAFIIFWVR

SEQ ID NO: 5 - DNA, 84 bp, synthetic construct
  source 1..84: mol_type = other DNA
ttttgggtgcttgtcgtggtgggtggcgtgctggcatgctactctttgctggtgaccgtggcctttattatcttttgggtgagg

SEQ ID NO: 6 - protein, 40 aa, synthetic construct
SKRSRLLHSDYMNMTPRRPGPTRKHYQPYAPPRDFAAYRS

SEQ ID NO: 7 - DNA, 120 bp, synthetic construct
  source 1..120: mol_type = other DNA
tccaagaggagtcggctgcttcactccgattacatgaacatgacccctaggcgacctggaccaacaagaaagcactaccagccatacgctccgccgagagactttgccgcatacagatcc

SEQ ID NO: 8 - protein, 240 aa, synthetic construct
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGVGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIK

SEQ ID NO: 9 - DNA, 720 bp, synthetic construct
  source 1..720: mol_type = other DNA
caggttcagctccagcagagcgggcccgaactcgaaaaaccaggagcaagtgtgaaaatctcctgtaaggcatctggctactccttcacaggatacacgatgaactgggtgaaacagtctcatggcaagtccctggagtggattggactgattacaccttataatggcgctagttcctacaaccagaaatttaggggtaaagctactctgacagtggacaaatcatcctctaccgcttacatggacctcctctccctgactagcgaagactccgctgtgtatttttgtgcccgggggggatacgatggcagaggatttgactattggggtcaggggactacagtgactgtatcatcaggcgttggaggaagtggtggtggaggaagcggagggggaggaagtgacatcgaactgacccagtcccctgctatcatgagcgcttccccaggcgaaaaagtcactatgacatgcagcgcctccagctccgtatcatatatgcactggtaccagcagaagtccggcacaagtcccaagaggtggatttatgacaccagtaagcttgcgtctggagtgcctggacgctttagtggttccggcagcggcaactcatactctctgactatcagcagtgttgaagccgaagatgacgccacctattattgtcagcagtggagtggctacccacttaccttcggcgccggaacaaagctggagatcaaa

SEQ ID NO: 10 - protein, 245 aa, synthetic construct
QVQLVQSGAEVKRPGASVQVSCRASGYSINTYYMWVRQAPGAGLEWMGVINPSGVTSYAQKFQGRVTLTNDTSTNTVYMQLNSLTSADTAVYYCARWALWGDFGMDVWGKGTLVTVSSGGGGSGGGGSGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIK

SEQ ID NO: 11 - DNA, 735 bp, synthetic construct
  source 1..735: mol_type = other DNA
caggtgcagttggtgcaatccggggcagaagtaaagcgacccggcgcaagtgtacaggtcagttgtcgggccagcggttatagtatcaatacatactatatgtgggtgagacaagcccctggggctgggcttgaatggatgggagtaattaacccctcaggcgtaacttcctatgctcagaaattccagggccgagtcacacttaccaacgacacgagcaccaacaccgtgtacatgcagctgaatagtctcacctccgccgatacagctgtttactattgcgctagatgggccctttggggcgatttcggcatggatgtctgggggaaaggcacactcgtgaccgtgtcatcaggcggtggtggttctggaggaggtggctctggaggtgggggttctggaggaggtggatcagatattcaaatgacacagtcaccaagcaccctgagcgcttcaattggggatcgcgtgacaatcacttgccgagcctccgaggggatctaccactggctcgcttggtaccaacagaagcccggcaaggctccgaaactcctgatctataaggcatctagtctcgcttcaggcgctcctagcagattttcaggatctggcagtgggacggactttactttgaccatttctagcttgcagcccgatgacttcgccacctactattgtcagcagtattctaactatccactcaccttcggtggcggaacaaagctggaaatcaag

SEQ ID NO: 12 - protein, 113 aa, synthetic construct
QVQLVQSGGGLVQPGGSLRLSCAASDFDFAAYEMSWVRQAPGQGLEWVAIISHDGIDKYYTDSVKGRFTISRDNSKNTLYLQMNTLRAEDTATYYCLRLGAVGQGTLVTVSSS

SEQ ID NO: 13 - DNA, 339 bp, synthetic construct
  source 1..339: mol_type = other DNA
caagtccagctcgtgcagagtggagggggactggtgcaacctggaggaagtctgcgcctgagctgtgctgcctctgatttcgatttcgcagcttatgagatgtcatgggtgaggcaggccccaggacaaggtctggagtgggtggctattatttcccacgatggaattgataaatattacaccgactctgttaaaggacggttcaccatctcccgggacaacagcaaaaacacgctttatctgcagatgaatacactgagagccgaggacaccgccacttattattgtttgaggctgggggcagtgggacaagggacactggtaaccgtgtcttcctct

SEQ ID NO: 14 - protein, 45 aa, synthetic construct
TTTPAPRPPTPAPTIASQPLSLRPEACRPAAGGAVHTRGLDFACD

SEQ ID NO: 15 - DNA, 135 bp, synthetic construct
  source 1..135: mol_type = other DNA
acaactacacctgcccctagacctcccacaccagcaccaacaattgcatctcagccgctgtcactgcgaccagaggcatgcagaccagccgctggtggcgctgttcataccagaggattggacttcgcatgcgat

SEQ ID NO: 16 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 17 - DNA, 339 bp, synthetic construct
  source 1..339: mol_type = other DNA
agggtaaaattctccagaagtgcagatgcccccgcttatcagcagggccaaaatcagctctataatgaactgaatcttgggcgccgcgaggaatacgatgtgctcgacaaacgacggggtcgcgatccagaaatgggaggcaagcctcagaggcgcaagaacccgcaggaaggactctacaatgagttgcagaaggacaagatggcagaagcatacagtgagattgggatgaaaggcgaaaggaggaggggaaaggggcatgatgggttgtatcagggcttgtctactgctacaaaggacacgtatgacgcactccacatgcaggctctgcccccaaga

SEQ ID NO: 18 - protein, 503 aa, synthetic construct
MALPVTALLLPLALLLHAARPNWSHPQFEKGSQVQLVQSGAEVKRPGASVQVSCRASGYSINTYYMWVRQAPGAGLEWMGVINPSGVTSYAQKFQGRVTLTNDTSTNTVYMQLNSLTSADTAVYYCARWALWGDFGMDVWGKGTLVTVSSGGGGSGGGGSGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 19 - DNA, 1512 bp, synthetic construct
  source 1..1512: mol_type = other DNA
atggctcttccagtgaccgcattgctgcttccattggctttgctgctgcacgccgcaaggcccaattggtcacatcctcagttcgagaaagggtcacaggtgcagttggtgcaatccggggcagaagtaaagcgacccggcgcaagtgtacaggtcagttgtcgggccagcggttatagtatcaatacatactatatgtgggtgagacaagcccctggggctgggcttgaatggatgggagtaattaacccctcaggcgtaacttcctatgctcagaaattccagggccgagtcacacttaccaacgacacgagcaccaacaccgtgtacatgcagctgaatagtctcacctccgccgatacagctgtttactattgcgctagatgggccctttggggcgatttcggcatggatgtctgggggaaaggcacactcgtgaccgtgtcatcaggcggtggtggttctggaggaggtggctctggaggtgggggttctggaggaggtggatcagatattcaaatgacacagtcaccaagcaccctgagcgcttcaattggggatcgcgtgacaatcacttgccgagcctccgaggggatctaccactggctcgcttggtaccaacagaagcccggcaaggctccgaaactcctgatctataaggcatctagtctcgcttcaggcgctcctagcagattttcaggatctggcagtgggacggactttactttgaccatttctagcttgcagcccgatgacttcgccacctactattgtcagcagtattctaactatccactcaccttcggtggcggaacaaagctggaaatcaagacaacaactcctgctccacgaccacccacacctgctccaactattgcctctcaacccctttccctgcggccagaggcttgtagacctgctgctggaggtgccgtgcatactagaggcttggacttcgcctgcgacttttgggtgcttgtcgtggtgggtggcgtgctggcatgctactctttgctggtgaccgtggcctttattatcttttgggtgaggtccaagaggagtcggctgcttcactccgattacatgaacatgacccctaggcgacctggaccaacaagaaagcactaccagccatacgctccgccgagagactttgccgcatacagatcccgcgtgaagttctccaggtccgcagatgctccagcctatcagcagggccagaatcagctctacaacgaactgaatctcggccgccgggaggaatatgatgtgctcgacaaacgcagaggtagagacccagaaatgggcggaaaacctcagcgaagaaaaaaccctcaagaggggctgtacaacgagctgcagaaagacaagatggccgaagcatacagcgaaataggaatgaagggggaaagaaggcggggcaaaggtcatgatggtttgtatcagggcctgtcaacagccacaaaggatacttatgatgctctgcacatgcaggccttgccacctaggtga

SEQ ID NO: 20 - protein, 498 aa, synthetic construct
MALPVTALLLPLALLLHAARPNWSHPQFEKGSQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGVGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 21 - DNA, 1497 bp, synthetic construct
  source 1..1497: mol_type = other DNA
atggcattgccagttacagcacttttgctcccacttgccctgctgctgcatgccgctagacccaattggagtcacccacagtttgagaagggctctcaggttcagctccagcagagcgggcccgaactcgaaaaaccaggagcaagtgtgaaaatctcctgtaaggcatctggctactccttcacaggatacacgatgaactgggtgaaacagtctcatggcaagtccctggagtggattggactgattacaccttataatggcgctagttcctacaaccagaaatttaggggtaaagctactctgacagtggacaaatcatcctctaccgcttacatggacctcctctccctgactagcgaagactccgctgtgtatttttgtgcccgggggggatacgatggcagaggatttgactattggggtcaggggactacagtgactgtatcatcaggcgttggaggaagtggtggtggaggaagcggagggggaggaagtgacatcgaactgacccagtcccctgctatcatgagcgcttccccaggcgaaaaagtcactatgacatgcagcgcctccagctccgtatcatatatgcactggtaccagcagaagtccggcacaagtcccaagaggtggatttatgacaccagtaagcttgcgtctggagtgcctggacgctttagtggttccggcagcggcaactcatactctctgactatcagcagtgttgaagccgaagatgacgccacctattattgtcagcagtggagtggctacccacttaccttcggcgccggaacaaagctggagatcaaaacaactacaccagctccgagacccccaacaccagcacctacaattgcaagccagccgctttctttgagacctgaagcttgtagaccagcagcagggggcgccgtacatactagagggcttgattttgcctgcgacttctgggtgttggtcgtcgtcggcggcgttttggcgtgctacagccttctggttacagtggcgttcatcatcttttgggtgaggtcaaaaagatcccgcctcctgcatagtgattacatgaacatgacccccagaagaccagggcccactagaaaacattaccagccatacgctccaccaagggacttcgctgcgtacagatcaagggtgaaattttcacggagtgccgacgctcctgcttaccaacagggtcagaatcagctgtataatgagttgaatctgggccgaagggaggaatatgatgtgttggataaacgcagagggagggatcctgaaatgggcggcaagccacagagaagaaaaaatcctcaggagggcctttataacgagctgcagaaagacaaaatggctgaggcttactctgagattggaatgaaaggagagcgaaggcgggggaaaggacatgacggcctgtaccaaggcctctcaacagccaccaaagatacttacgatgctctgcacatgcaggcccttccaccaagatga

SEQ ID NO: 22 - protein, 371 aa, synthetic construct
MALPVTALLLPLALLLHAARPNWSHPQFEKGSQVQLVQSGGGLVQPGGSLRLSCAASDFDFAAYEMSWVRQAPGQGLEWVAIISHDGIDKYYTDSVKGRFTISRDNSKNTLYLQMNTLRAEDTATYYCLRLGAVGQGTLVTVSSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 23 - DNA, 1116 bp, synthetic construct
  source 1..1116: mol_type = other DNA
atggctcttccagttacagctctgcttttgcccttggctctcctgcttcacgcagctcggcctaattggtcacatccccagtttgaaaagggctcccaagtccagctcgtgcagagtggagggggactggtgcaacctggaggaagtctgcgcctgagctgtgctgcctctgatttcgatttcgcagcttatgagatgtcatgggtgaggcaggccccaggacaaggtctggagtgggtggctattatttcccacgatggaattgataaatattacaccgactctgttaaaggacggttcaccatctcccgggacaacagcaaaaacacgctttatctgcagatgaatacactgagagccgaggacaccgccacttattattgtttgaggctgggggcagtgggacaagggacactggtaaccgtgtcttcctctaccactacaccagcacccagaccacctacacctgcccctactatcgcttcacagcccctctcactcaggcccgaagcatgtagacctgctgccggcggagcagttcataccagagggctggatttcgcctgtgatttctgggtgttggtggtagtcggaggggtgctggcctgttacagcctcctggtgacggtggcgtttatcatcttctgggtgcgctccaaaagaagcagactgctgcacagcgactacatgaatatgacacctcggagaccggggccaactagaaagcactaccagccgtatgcaccccctcgggatttcgcagcatataggtcccgcgtcaagttctccaggtccgccgatgcgcccgcatatcaacaggggcagaaccaactgtataacgaactgaatcttggtagaagggaagaatacgacgttctggataaacgcaggggcagagatcctgagatggggggaaagccccaacgacggaaaaacccacaggagggcctgtacaacgagctgcagaaggataaaatggcggaggcttactccgagatagggatgaagggcgagagacgcagggggaagggacatgacgggttgtatcaggggctctctacagccacgaaggatacttatgatgctctgcatatgcaggctctgcctccacgatga

SEQ ID NO: 24 - DNA, 6217 bp, synthetic construct
  source 1..6217: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatagatacgaattaattctaccacccctctccagccactaagccagttgctccctcggctgacggctgcacgcgaggcctccgaacgtcttacgccttgtggcgcgcccgtccttgtcccggggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgacggatccgctagcgtttaaacgcggccgcgaattcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

(19) United States
(12) Patent Application Publication
(10) Pub. No.: US 2026/0015431 A1
(43) Pub. Date: Jan. 15, 2026

(54) CAR-iNKT CELL TECHNOLOGY EFFECTIVE IN KILLING CHOLANGIOCARCINOMA

(71) Applicant: Beijing Gene Key Life Technology Co., Ltd., Daxing District, Beijing (CN)

(72) Inventors: Jun LU, Beijing (CN); Xianghe LIU, Beijing (CN); Jikai FENG, Beijing (CN)

(21) Appl. No.: 18/559,529

(22) PCT Filed: Mar. 16, 2023
(86) PCT No.: PCT/CN2023/081894

§ 371 Date: Nov. 7, 2023

(30) Foreign Application Priority Data
Jan. 20, 2023 (CN) 202310087774.1

(51) Int. Cl. C07K 16/30; A61K 40/15; A61K 40/31; A61K 40/42; A61P 35/00; C07K 14/705; C07K 14/725; C12N 5/0783

(52) U.S. Cl. CPC C07K 16/30; A61K 40/15; A61K 40/31; A61K 40/4255; A61P 35/00; C07K 14/7051; C07K 14/70517; C07K 14/70521; C12N 5/0646; C07K 2317/53; C07K 2319/03; C12N 2510/00

(57) ABSTRACT

The present application provides a MSLN-containing chimeric antigen receptor, iNKT cells transduced by the chimeric antigen receptor and use of the chimeric antigen receptor and the INKT cells in treatment of liver cancer, particularly cholangiocarcinoma. The present application utilizes the characteristic that iNKT cells can home and colonize the liver, and selects a proper MSLN antibody sequence, thereby realizing high tumor killing efficiency and CAR-iNKT cell proliferation speed; the Anti-MSLN CAR-INKT cell can effectively infiltrate into the liver tumor part, greatly improve the curative effect, reduce the recurrence and alleviate the toxic and side effects.

### CROSS-REFERENCE TO RELATED APPLICATIONS

The present application claims priority of the Chinese Patent Application number 202310087774.1, filed on Jan. 20, 2023 before the China National Intellectual Property Administration, with the title of “CAR-INKT CELL TECHNOLOGY EFFECTIVE IN KILLING CHOLANGIOCARCINOMA”, which is hereby incorporated by reference in its entirety.

### FIELD OF THE INVENTION

The present application relates to the technical field of cancer immunotherapy. Specifically, the present application provides a chimeric antigen receptor comprising MSLN, iNKT cells transduced with the chimeric antigen receptor, and uses thereof in the treatment of liver cancer, particularly cholangiocarcinoma.

### BACKGROUND OF THE INVENTION

According to WHO statistics, in 2020, there were 900,000 new cases of liver cancer in the world^{[}1], and 410,000 new cases of liver cancer in China^{[}2], accounting for 45% of the incidence of liver cancer in the world; in 2020, there were 830,000 deaths due to liver cancer in the world^{[}1], and 390,000 deaths due to liver cancer in China^{[}2], accounting for 46% of the deaths due to liver cancer in the world. Cholangiocarcinoma accounts for about 10% of all liver cancers^{[}3].

Cholangiocarcinoma is a kind of tumor originating from the epithelial layer of intrahepatic or extrahepatic bile ducts. Cholangiocarcinoma is not easily detected in its early stages and has a poor outcome after diagnosis, with a five-year survival rate of only 10%^{[4]}. Approximately only one-third of patients with cholangiocarcinoma after diagnosis are suitable for surgical treatment^{[5]}. Treatment methods for cholangiocarcinoma include surgical resection of the tumour, liver transplantation, radiotherapy, chemotherapy, local therapy, molecular targeted therapy, and immunotherapy^{[6]}. The existing treatment methods cannot meet the clinical requirements, and a safer and more effective treatment method is urgently needed for cholangiocarcinoma.

The chimeric antigen receptor technology has achieved a significant therapeutic effect in treating blood tumor, with Kymriah^{[7]} and Yescata^{[8]} receiving FDA approval in 2017 for the treatment of B-cell acute lymphoblastic leukaemia and diffuse large B-cell lymphoma. China also approved the first chimeric antigen receptor cell product for hematological tumor therapy in June 2021^{[9]}.

Mesothelin (MSLN) is a glycosyl-phosphatidyl inositol-anchored protein expressed on the surface of cell membranes, under normal physiological conditions expressed in the pleura, peritoneum, pericardium and tunica vaginalis of males, and low expressed in the epithelial layer of ovaries, fallopian tubes and testis^{[10]}. The human MSLN gene is located on chromosome 16 and codes for a precursor protein of 628 amino acids with a molecular weight of approximately 71 kDa^{[14]}. The MSLN precursor protein is spliced at Arg295 to the N-terminal soluble protein megakaryocyte-potentiating factor (MPF) and glycosylphosphatidylinositol anchored MSLN that binds to the cell membrane surface^{[20]}.

Compared with limitedly expression in mesothelial cells under normal conditions, MSLN is over expressed in various tumors, including malignant mesothelioma^{[11]}, ovarian cancer^{[12]}, triple negative breast cancer^{[13]}, prostate cancer^{[14]}, lung cancer^{[15]}, gastric cancer^{[16]}, endometrial cancer^{[17]}, cervical cancer^{[18]}, cholangiocarcinoma and the like, and is an ideal target for targeted therapy of a chimeric antigen receptor technology.

Invariant natural killer T cells (INKT) are a unique subset of thymic derived T cells with CD1d restriction and surface receptors that simultaneously express T cell and natural killer cell (NK) lineage characteristics. They share the common biological characteristics of T cells and NK cells and play an important role in bridging innate and adoptive immunity. In human iNKT cells, Vα24-Jα18 forms the TCRα chain, which subsequently forms TCR with the Vβ11 TCRβ chain^{[21]}.

iNKT cells differentiate into at least three effector subsets in the thymus, similar to the subsets of CD4+ T helper cells, but also to the subsets of innate lymphoid cells (ILCs)^{[22-24]} Functional iNKT cell subsets are distinguished on the basis of the expression of different cell surface markers and characteristic transcription factors. NKT1 cells are similar to Th1 cells and ILCls as they all highly express the transcription factor T-bet and all secrete IFN-γ upon activation. NKT1 cells also exhibit greater cytotoxicity than other iNKT cell subsets. NKT1 cells differ from Th1 cells or ILC1s in that, in addition to producing IFN-γ through TCR activation, they also produce factors such as IL-4. Similar to Th2 cells, NKT2 cells secrete cytokines including IL-4 and IL-13, while NKT17 cells are similar to Th17 cells in terms of cytokine secretion^{[25-27]}.

Different subsets of iNKT cells are enriched in different tissues. NKT1 cells are highly enriched in the liver, whereas NKT17 cells are mainly located in lymph nodes, skin and lungs, with small numbers in the spleen^{[}28]. NKT2 cells are located in several sites, including the lungs and the spleen, but they are particularly abundant in mesenteric lymph nodes^{[28]}. In peripheral lymph nodes, iNKT cells can be rapidly activated and may play a key role in the fight against pathogenic agents^{[29]}.

The immunotherapeutic research on NKT cells started late, with low levels in vivo, and the selection of their specific antibodies is relatively limited. Currently, there are only relevant researches on designing antibodies against GD2, CD19, CSPG4, and BCMA, and there are fewer types of specific antibodies available for reference, which makes it relatively more difficult to select and design them.

In AU2020264343A1 and CN112574953A, which are close to the present application, T cells are genetically modified to express chimeric receptors targeting MSLN antigens, wherein the CARs include MSLN antigen-binding structural domains, transmembrane structural domains, co-stimulatory signaling regions, and CD3ζ signaling structural domains, and exhibit a killing effect on cancer cells carrying MSLN antigen. However, considering that ordinary T cells cannot effectively infiltrate into solid tumors, the tumor microenvironment of solid tumors is hypoxic and acidic, which is very unfavorable to the expansion and long-term survival of CAR-T cells, thus seriously affecting the efficacy of CAR-T cells.

### SUMMARY OF THE INVENTION

To address the above problems, the applicant took advantage of the characteristics of iNKT cells homing to the liver and their non-specific killing function, and genetically modified them to carry chimeric antigen receptors that can bind MSLN antigens, so that the anti-MSLN-CAR-iNKT cells can effectively perform specific killing of cholangiocarcinoma cells carrying MSLN antigens; and compared CAR molecules constituted by different scFvs, to select CAR molecules that are more favorable for binding MSLN antigen on the surface of cancer cells and killing cancer cells. Specifically:

The specific scFv targeting MSLN was introduced into the chimeric antigen receptor (CAR) molecule, so that it can specifically bind to MSLN antigen specifically expressed by cholangiocarcinoma cells, and at the same time, the co-stimulatory molecules CD28 intracellular signal domain and CD3ζ intracellular signal domain were introduced into the CAR molecule, so that it can stimulate the proliferation of iNKT cells and the secretion of IFN-γ, granzymes and so on, which can be used for the killing of the cancer cells, and the killing effects of CAR molecules constituted by different scFvs on the cancer cells expressing MSLN antigen were compared, so as to select a CAR molecule with a better anticancer effect.

The inventors, after creative labor, have continuously carried out the design of amino acid sequences as well as sequence permutations and screenings, and conducted random screening tests and targeting function verification of nearly dozens of CAR molecular sequences (such as constructing viral vectors, as well as further infecting iNKT cells, obtaining modified iNKT cells, and detecting the in vitro killing activity of the resulting modified iNKT cells, and other tests), and then made sequence adjustments based on the comparison of the results of multiple random combinations, and finally screened out the sequences with the best results, and obtained the chimeric antigen receptor sequences of anti-human MSLN single-chain antibodies targeting the human MSLN protein at high efficiencies in the present application, and the functional variants thereof.

In one aspect, the present application provides a chimeric antigen receptor comprising a MSLN single-chain antibody, a spacer or hinge region, a transmembrane region, an intracellular co-stimulatory domain, and a signal region, linked in sequence.

Optionally, the chimeric antigen receptor comprises a CD8α signal peptide, a MSLN single-chain antibody, a CD8α hinge region, a CD28 transmembrane region, a CD28 co-stimulatory region, and a CD3ζ signal region, linked in sequence.

Optionally, the MSLN single-chain antibody has an amino acid sequence selected from the group consisting of SEQ ID NO. 8, SEQ ID NO. 10 and SEQ ID NO. 12.

Optionally, the MSLN single-chain antibody has a nucleotide sequence selected from the group consisting of SEQ ID NO. 9, SEQ ID NO. 11, and SEQ ID NO. 13.

Optionally, the amino acid sequence of the MSLN single-chain antibody is SEQ ID No. 8.

Optionally, the amino acid sequence of the CD8α signal peptide is SEQ ID NO. 2, the amino acid sequence of the CD8α hinge region is SEQ ID NO. 14, the amino acid sequence of the CD28 transmembrane region is SEQ ID NO. 4, the amino acid sequence of the CD28 co-stimulatory region is SEQ ID NO. 6, and the amino acid sequence of the CD3ζ signal region is SEQ ID NO. 16.

Optionally, the nucleotide sequence of the CD8α signal peptide is SEQ ID NO. 3, the nucleotide sequence of the CD8α hinge region is SEQ ID NO. 15, the nucleotide sequence of the CD28 transmembrane region is SEQ ID NO. 5, the nucleotide sequence of the CD28 co-stimulatory region is SEQ ID NO. 7, and the nucleotide sequence of the CD3ζ signal region is SEQ ID NO. 17.

Optionally, the chimeric antigen receptor has an amino acid sequence selected from the group consisting of SEQ ID NO. 18, SEQ ID NO. 20, and SEQ ID NO. 22.

Optionally, the chimeric antigen receptor has a nucleotide sequence selected from the group consisting of SEQ ID NO. 19, SEQ ID NO. 21, and SEQ ID NO. 23.

In another aspect, the present application provides a vector carrying the chimeric antigen receptor described above; optionally, the vector is a pLV300 vector.

Optionally, the nucleotide sequence of the pLV300 vector is SEQ ID NO. 24.

In another aspect, the present application provides an immune cell which transduces the chimeric antigen receptor described above.

Optionally, the immune cell is a T cell, an NK cell or an iNKT cell.

Optionally, the immune cell is an iNKT cell.

In another aspect, the present application provides use of the chimeric antigen receptor, the vector, or the immune cell described above in the manufacture of a medicament for treating cancer.

In another aspect, the present application provides a method for treating cancer, wherein the chimeric antigen receptor, the vector or the immune cell is used.

Optionally, the cancer is a MSLN-overexpressing cancer.

Optionally, the cancer is liver cancer.

Optionally, the cancer is cholangiocarcinoma.

In another aspect, the present application provides a transduction system comprising the vector described above.

Optionally, the transduction system is a viral transduction system and a non-viral transduction system.

Optionally, the transduction system is a lentiviral transduction system.

The present application includes a nucleic acid expression vector comprising one or more of the above elements and used for constructing chimeric antigen receptor proteins expressed on the surface of T cells, NK cells or NKT cells. Various commercially available vectors may be used as desired, or the vectors may be constructed according to conventional techniques in the field of molecular biology. In one specific embodiment, the vector used in the present application is a lentiviral plasmid vector pLV300. The plasmid belongs to the fourth generation of self-inactivating lentiviral vector system that has four plasmids, i.e., a packaging plasmid encoding the protein Gag/Pol, a packaging plasmid encoding the Rev protein, an envelope plasmid encoding the VSV-G protein, and an empty vector pLV300, which can be used for recombinant and introduction of a target nucleic acid sequence, i.e. a nucleic acid sequence encoding the chimeric antigen receptor protein. The expression of the chimeric antigen receptor protein is regulated by a pGK-300 promoter in the vector pLV300.

The present application includes a virus comprising the vectors described above, including but not limited to a lentivirus, a retrovirus, an adenoviruses, an adeno-associated virus, and the like. The virus of the present application includes a packaged infectious virus, and also includes a virus to be packaged that contains components necessary for packaging into an infectious virus. Other virus known in the art for transfecting T cells, NK cells or NKT cells and their corresponding plasmid vectors may also be used in the present application. In one embodiment of the present application, the virus is a lentivirus comprising the pLV300-anti-GPC3 CAR recombinant vector described above.

The present application includes a transgenic T-lymphocyte, NK cell or iNKT cell, which is transduced with a nucleic acid of the present application or is transduced with a recombinant plasmid of the present application containing the nucleic acid, or a viral system comprising the plasmid. Nucleic acid transduction methods conventional in the art, including both non-viral and viral transduction methods, can be used in the present application. Non-viral based transduction methods include electroporation and transposon methods. Recently, Amaxa has developed the nucleofector, which is capable of directly introducing exogenous genes into the nucleus for efficient transduction of target genes. In addition, based on the Sleeping Beauty system or PiggyBac and other transposon systems, the efficiency of transduction is much higher than that of ordinary electroporation, and the combination application of nucleofector and Sleeping Beauty system has been reported, which is able to achieve both higher transduction efficiency and site-directed integration of target genes. In one embodiment of the application, the transduction method to achieve chimeric antigen receptor gene modified iNKT cells is a lentivirus-based transduction method. The method has the advantages of high transduction efficiency, stable expression of exogenous genes, shortened time for in vitro culture of iNKT lymphocytes to reach clinical level numbers, and the like. The nucleic acid introduced by lentivirus transfection is expressed on the cell membrane surface of iNKT by transcription and translation. In vitro cytotoxicity experiments on various cultured tumor cells have shown that the transgenic iNKT cells with the chimeric antigen receptor expressed on the surface of the present application have high-specificity tumor cell killing effect (also called cytotoxicity). Thus, the nucleic acid encoding the chimeric antigen receptor protein, the plasmid comprising the nucleic acid, the virus comprising the plasmid, and the transgenic iNKT cell, T lymphocyte, or NK cell transfected with the above nucleic acid, plasmid, or virus of the present application can be effectively used for immunotherapy of tumors.

The nucleic acid of the present application may be in the form of DNA or in the form of RNA. The form of DNA includes cDNA, genomic DNA or artificially synthesized DNA. The DNA may be single-stranded or double-stranded. The DNA may be a coding strand or a non-coding strand. The nucleic acid codons encoding the amino acid sequences of the chimeric antigen receptor proteins of the present application may be degenerate, i.e., a plurality of degenerate nucleic acid sequences encoding the same amino acid sequence are included within the scope of the present application. Degenerate nucleic acid codons encoding corresponding amino acids are well known in the art. The present application also relates to variants of the above polynucleotides which encode a polypeptide having the same amino acid sequence as the present application or fragments, analogues and derivatives of the polypeptide. The variant of the polynucleotide may be a naturally occurring allelic variant or a non-naturally occurring variant. These nucleotide variants include substitution variants, deletion variants and insertion variants. As is known in the art, an allelic variant is a substitution form of a polynucleotide, which may be a substitution, deletion, or insertion of one or more nucleotides, without substantially changing the function of the encoded polypeptide.

The MSLN-overexpressing cancer includes but is not limited to malignant mesothelioma, ovarian cancer, triple negative breast cancer, prostate cancer, lung cancer, gastric cancer, endometrial cancer, cervical cancer, cholangiocarcinoma and the like.

The MSLN-overexpressing refers to a condition that the content of the MSLN-associated nucleic acid or protein detected by various detection methods, including but not limited to, detection methods at the molecular level, and detection methods at the protein level, or other indications are higher than normal level.

### BRIEF DESCRIPTION OF THE DRAWINGS

FIG. 1 is a structural schematic diagram of an anti-MSLN CAR;

FIG. 2 is a structural schematic diagram of a pLV300 lentiviral vector;

FIG. 3 is a graph of iNKT total cell proliferation;

FIG. 4 shows the proportion of CD8+ CAR+iNKT cell subsets in anti-MSLN CAR-iNKT cell culture at different times;

FIG. 5 shows the proportion of CD4+ CAR+iNKT cell subsets in anti-MSLN CAR-iNKT cell culture at different times;

FIG. 6 shows the proportion of CD4−CD8−CAR+iNKT cell subsets in anti-MSLN CAR-iNKT cell culture at different times;

FIG. 7 shows the killing effect of anti-MSLN CAR-INKT on cholangiocarcinoma cells KMCH at different effector: target ratios;

FIG. 8 shows the IFN-γ content in the cell culture supernatant after 24 hours of co-culture of anti-MSLN CAR-iNKT cells and cholangiocarcinoma cells KMCH;

FIG. 9 shows the IL-2 content in the cell culture supernatant after 24 hours of co-culture of anti-MSLN CAR-INKT cells and cholangiocarcinoma cells KMCH.

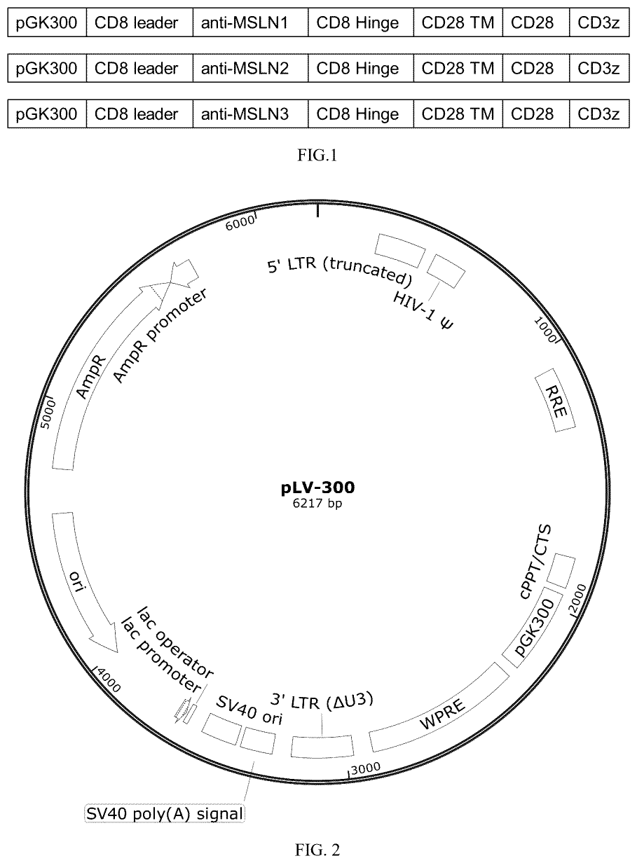

### DETAILED DESCRIPTION OF THE INVENTION

### EXAMPLE 1

### Preparation of Lentiviruses Containing Chimeric Antigen Receptor Molecules

The HEK-293T cells were passaged. After the cells grew to 60%-70% confluency, the expression vectors containing the CAR molecules together with the packaging plasmid were transfected into HEK-293T cells with pEI reagent, with fresh medium being changed 4 hours after transfection. Cell culture supernatants were collected after 48-72 hours of transfection. The supernatant was ultra-centrifuged to concentrate the packaged lentivirus containing CAR molecules. The virus titer of the concentrated lentivirus was determined, and the lentivirus was frozen at −80° C. until use.

### EXAMPLE 2

### Effect of Anti-MSLN CAR Molecules on Total iNKT Cell Proliferation and CAR-INKT Cell Differentiation

After isolating iNKT cells from human peripheral blood mononuclear cells using anti-iNKT mircobeads, cells were seeded in a 24-well plate at 2×10⁵ cells per well, X-VIVO complete culture medium (containing 100 IU/ml and 100 ng/ml α-Galcer) was added per well to culture for 48 hours. The cells in each well were collected, and centrifuged at 400×g for 5 minutes. After the supernatant was discarded, fresh X-VIVO complete culture medium was added to re-suspend the cells, and the cells were re-seeded in a 24-well plate, and different lentiviruses containing anti-MSLN1 CAR, anti-MSLN2 CAR, and anti-MSLN3 CAR were added respectively into each well of cells to infect them. After 24 hours, the cells in each well were collected in centrifuge tubes, centrifuged at 400×g for 5 minutes, counted, and cultured by adding fresh X-VIVO complete culture medium at 5×10⁵ cells/ml, and changing the medium every 48 hours. When cultured to day 7, day 14, and day 21, respectively, samples were taken, counted and flow-tested to confirm the expression of the chimeric antigen receptor molecule.

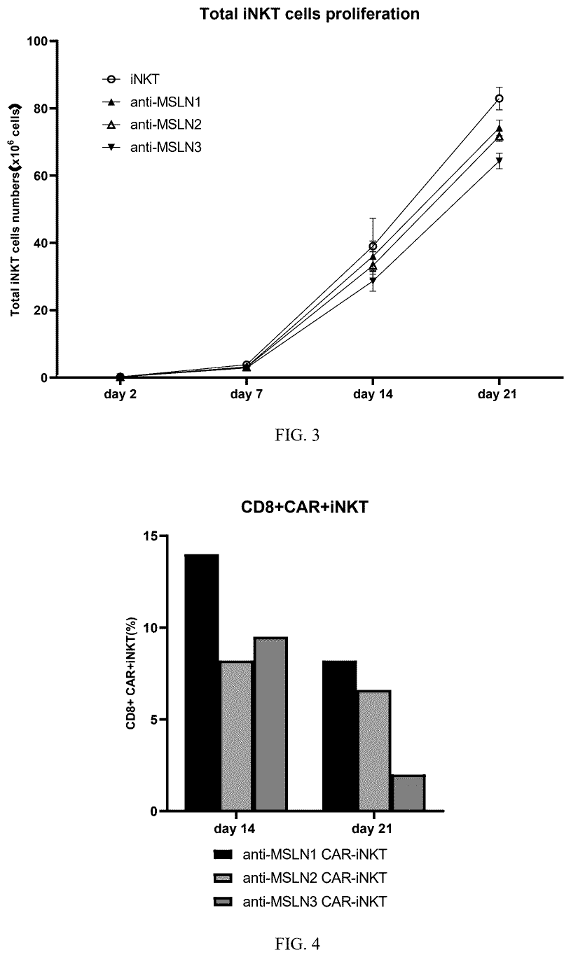

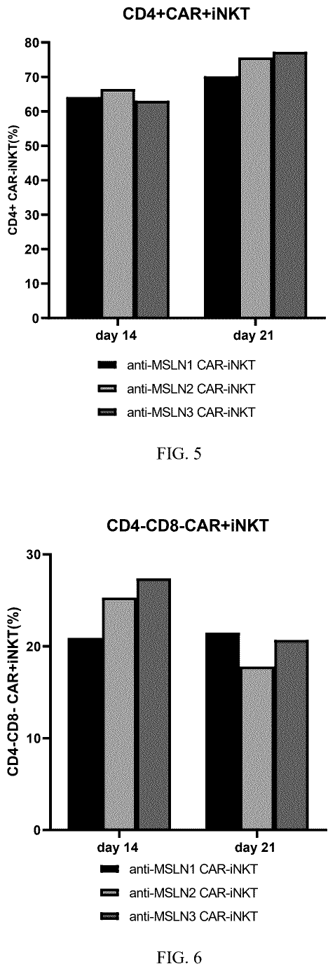

The results show that: different anti-MSLN CAR molecules have influence on the proliferation and differentiation of iNKT cells, anti-MSLN1 CAR and anti-MSLN2 CAR are more beneficial to cell growth than anti-MSLN3 CAR molecules after being transferred into iNKT cells (FIG. 3), and anti-MSLN1 CAR and anti-MSLN2 CAR are more beneficial to the differentiation of CAR-INKT cells into CD8+ CAR+iNKT cells than anti-MSLN3 CAR molecules after being transferred into iNKT cells (FIGS. 4-6).

### EXAMPLE 3

### Effect of Different Anti-MSLN CAR Molecules on the Tumor Killing Ability of CAR-INKT Cells

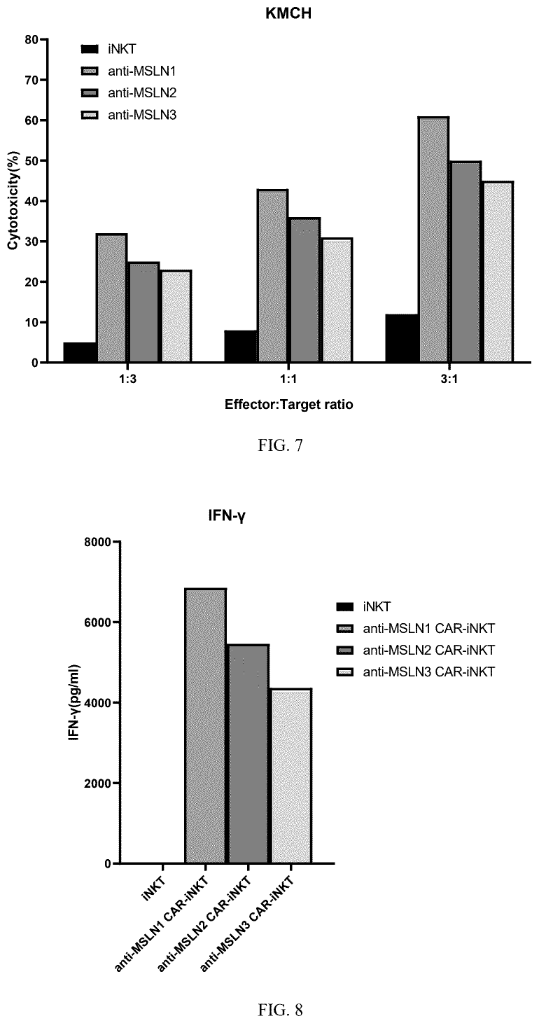

When the CAR-iNKT cells of different groups were cultured until day 21, 0.33×10⁵ CAR-iNKT cells, 1×10⁵ CAR-INKT cells, 3×10⁵ CAR-INKT cells, and 1×10⁵ KMCH cells (KMCH is cholangiocarcinoma cell line with positive Mesothelin expression; mixed incubation with fluorochrome CFSE for 10 minutes) were co-cultured for 6 hours at the effector: target ratios of 1:3, 1:1, and 3:1, respectively, and then the culture supernatants of each group were taken and measured for fluorescence intensity in the supernatants using a microplate reader (the higher the killing ability of CAR-iNKT cells, the more CFSE released in KMCH cells, the higher the fluorescence intensity), and the killing efficiency of CAR-iNKT cells of each group was calculated (FIG. 7). The results show that: three anti-MSLN CAR-INKT cells can effectively kill KMCH cells, but the anti-MSLN1 CAR-iNKT cells have the strongest killing ability.

### EXAMPLE 4

### Comparison of Secretion of IFN-γ And IL-2 by Different Anti-MSLN CAR-Inkt Cells Co-Incubated with MSLN-Expressing KMHC Cells

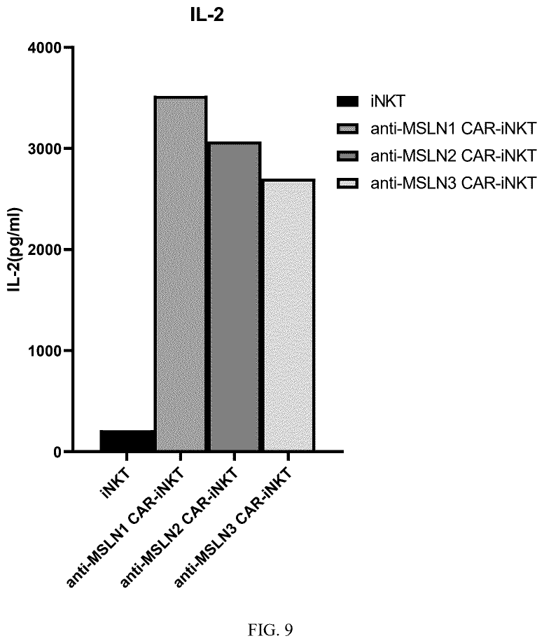

After each group of CAR-iNKT cells were cultured to day 21, according to the effector: target ratio being 3:1, after each group of CAR-iNKT cells (each group of 3×10⁵ CAR-iNKT cells) was co-cultured with 1×10⁵ KMHC cells in 0.5 ml X-VIVO (without IL-2 and α-GalCer) for 24 hours, the cells were collected, centrifuged at 400×g for 5 minutes, the supernatant was collected, and the contents of IFN-γ and IL-2 in the supernatant were measured by ELISA method. The results show that the anti-MSLNI CAR-INKT cell has the strongest capacity of secreting IFN-γ and IL-2, which is consistent with the strong proliferation capacity and the killing capacity of the anti-MSLN1 CAR-INKT cell to tumor (FIGS. 8-9).

It is obvious that the above-described examples of the present application are merely examples for clearly illustrating the present application and are not intended to limit the embodiments of the present application. Other variations and modifications in different forms can also be made for persons skilled in the art in light of the above description. This need not be, nor should it be exhaustive of all embodiments. Such obvious variations and modifications explicated from the spirit of the present application are within the scope of the present application.

### REFERENCES

1 https://gco.iarc.fr/today/data/factsheets/populations/900-world-fact-heets.pdf
2 https://gco.iarc.fr/today/data/factsheets/populations/160-china-fact-heets.pdf
3 Sahil Mittal 1, Hashem B El-Serag.Epidemiology of hepatocellular carcinoma: consider the population J Clin Gastroenterol. 2013 July;47 Suppl (0): S2-6. doi: 10.1097/MCG.0b013e3182872f29.
4 Everhart J E, Ruhl C E. Burden of digestive diseases in the United States part III: liver, biliary tract, and pancreas. Gastroenterology 2009 April; 136 (4): 1134e44. PubMed PMID: 19245868. Epub 2009 Feb. 28. eng.
5 Shaib Y H, Davila J A, Henderson L, McGlynn K A, El-Serag H B. Endoscopic and surgical therapy for intrahepatic cholangiocarcinoma in the United States: a population-based study. J Clin Gastroenterol 2007 NoveDec;41 (10): 911e7. PubMed PMID: 18090160. Epub 2007 Dec. 20. eng.
6 Khan S A, Davidson B R, Goldin R D, Heaton N, Karani J, Pereira S P, et al. Guidelines for the diagnosis and treatment of cholangiocarcinoma: an update. Gut 2012 December;61 (12): 1657e69. PubMed PMID: 22895392. Epub 2012 Aug. 17. eng.
7 https://www.fda.gov/media/106989/download
8 https://www.fda.gov/media/108458/download
9 https://www.nmpa.gov.cn/yaowen/ypjgyw/20210623094232187.html
10 Ordonez NG. Application of mesothelin immunostaining in tumor diagnosis. Am J Surg Pathol. 2003;27 (11): 1418-1428.
11 Ordonez N G. Value of mesothelin immunostaining in the diagnosis of mesothelioma. Mod Pathol. 2003; 16 (3): 192-197.
12 Scales S J, Gupta N, Pacheco G, et al. An antimesothelin-monomethyl auristatin e conjugate with potent antitumor activity in ovarian, pancreatic, and mesothelioma models. Mol Cancer Ther. 2014;13 (11): 2630-2640.
13 Li Y R, Xian R R, Ziober A, et al. Mesothelin expression is associated with poor outcomes in breast cancer. Breast Cancer Res Treat. 2014; 147 (3): 675-684.
14 Ho M, Bera T K, Willingham M C, et al. Mesothelin expression in human lung cancer. Clin Cancer Res. 2007;13 (5): 1571-1575.
15 Ho M, Bera T K, Willingham M C, et al. Mesothelin expression in human lung cancer. Clin Cancer Res. 2007;13 (5): 1571-1575.
16 Baba K, Ishigami S, Arigami T, et al. Mesothelin expression correlates with prolonged patient survival in gastric cancer. J Surg Oncol. 2012; 105 (2): 195-199.
17 Cheng W F, Huang C Y, Chang M C, et al. High mesothelin correlates with chemoresistance and poor survival in epithelial ovarian carcinoma. Br J Cancer. 2009;100 (7): 1144-1153.
18 Kawamata F, Kamachi H, Einama T, et al. Intracellular localization of mesothelin predicts patient prognosis of extrahepatic bile duct cancer. Int J Oncol.

2012;41 (6): 2109-2118.
19 Chang K, Pastan I. Molecular cloning of mesothelin, a differentiation antigen present on mesothelium, mesotheliomas, and ovarian cancers. Proc Natl Acad Sci U S A. 1996;93 (1): 136-140.
20 Shimizu A, Hirono S, Tani M, et al. Coexpression of MUC16 and mesothelin is related to the invasion process in pancreatic ductal adenocarcinoma. Cancer Sci. 2012; 103 (4): 739-746.
21 Crosby, C. M. and M. Kronenberg, Tissue-specific functions of invariant natural killer T cells. Nat Rev Immunol, 2018. 18 (9): p. 559-574.
22 Bennstein, S. B., Unraveling Natural Killer T-Cells Development. Front Immunol, 2017. 8: p. 1950.
23 Georgiev, H., et al., Distinct gene expression patterns correlate with developmental and functional traits of iNKT subsets. Nat Commun, 2016. 7: p. 13116.
24 Lee, Y. J., et al., Lineage-Specific Effector Signatures of Invariant NKT Cells Are Shared amongst gammadelta T, Innate Lymphoid, and Th Cells. J Immunol, 2016. 197 (4): p. 1460-70.
25 Lee, Y. J., et al., Steady-state production of IL-4 modulates immunity in mouse strains and is determined by lineage diversity of iNKT cells. Nat Immunol, 2013. 14 (11): p. 1146-54.
26 Verykokakis, M. and B. L. Kee, Transcriptional and epigenetic regulation of innate-like T lymphocyte development. Curr Opin Immunol, 2018. 51: p. 39-45.
27 Engel, I., et al., Innate-like functions of natural killer T cell subsets result from highly divergent gene programs. Nat Immunol, 2016. 17 (6): p. 728-39.
28 Lee, Y. J., et al., Tissue-Specific Distribution of iNKT Cells Impacts Their Cytokine Response. Immunity, 2015. 43 (3): p. 566-78.
29 Gaya, M., et al., Initiation of Antiviral B Cell Immunity Relies on Innate Signals from Spatially Positioned NKT Cells. Cell, 2018. 172 (3): p. 517-533 e20.

1. A chimeric antigen receptor, comprising a MSLN single-chain antibody, a spacer domain or hinge region, a transmembrane region, an intracellular co-stimulatory domain, and a signal region, linked in sequence.

2. The chimeric antigen receptor of claim 1, comprising a CD8α signal peptide, a MSLN single-chain antibody, a CD8α hinge region, a CD28 transmembrane region, a CD28 co-stimulatory region, and a CD3ζ signal region, linked in sequence.

3. The chimeric antigen receptor of claim 1, wherein the MSLN single-chain antibody has an amino acid sequence selected from the group consisting of SEQ ID NO. 8, SEQ ID NO. 10 and SEQ ID NO. 12.

4. The chimeric antigen receptor of claim 3, wherein the MSLN single-chain antibody has a nucleotide sequence selected from the group consisting of SEQ ID NO. 9, SEQ ID NO. 11, and SEQ ID NO. 13.

5. The chimeric antigen receptor of claim 3, wherein the amino acid sequence of the MSLN single-chain antibody is SEQ ID NO. 8.

6. The chimeric antigen receptor of claim 2, wherein the amino acid sequence of the CD8α signal peptide is SEQ ID NO. 2, the amino acid sequence of the CD8α hinge region is SEQ ID NO. 14, the amino acid sequence of the CD28 transmembrane region is SEQ ID NO. 4, the amino acid sequence of the CD28 co-stimulatory region is SEQ ID NO. 6, and the amino acid sequence of the CD3ζ signal region is SEQ ID NO. 16.

7. The chimeric antigen receptor of claim 6, wherein the nucleotide sequence of the CD8α signal peptide is SEQ ID NO. 3, the nucleotide sequence of the CD8α hinge region is SEQ ID NO. 15, the nucleotide sequence of the CD28 transmembrane region is SEQ ID NO. 5, the nucleotide sequence of the CD28 co-stimulatory region is SEQ ID NO. 7, and the nucleotide sequence of the CD3ζ signal region is SEQ ID NO. 17.

8. The chimeric antigen receptor of claim 1, wherein the chimeric antigen receptor has an amino acid sequence selected from the group consisting of SEQ ID NO. 18, SEQ ID NO. 20, and SEQ ID NO. 22.

9. The chimeric antigen receptor of claim 8, wherein the chimeric antigen receptor has a nucleotide sequence selected from the group consisting of SEQ ID NO. 19, SEQ ID NO. 21, and SEQ ID NO. 23.

10. A vector carrying the chimeric antigen receptor of claim 1.

11. The vector of claim 10, wherein the vector is pLV300 and the nucleotide sequence of pLV300 is SEQ ID NO. 24.

12. An immune cell transduced with the chimeric antigen receptor of claim 1.

13. The immune cell of claim 12, wherein the immune cell is a T cell, an NK cell, or an iNKT cell.

14. The immune cell of claim 12, wherein the immune cell is an iNKT cell.

15. (canceled)

16. A method of treating cancer, wherein the chimeric antigen receptor of claim 1 is used.

17. The method of claim 17, wherein the cancer is a MSLN-overexpressing cancer.

18. The method of claim 17, wherein the cancer is liver cancer.

19. The method of claim 18, wherein the cancer is cholangiocarcinoma.

20. A transduction system comprising the vector of claim 10.

21-25. (canceled)

26. A nucleic acid encoding the chimeric antigen receptor of claim 1, wherein the nucleotide sequence of the nucleic acid is selected from the group consisting of SEQ ID NO. 19, SEQ ID NO. 21 and SEQ ID NO. 23.
